(19) United States
(12) Patent Application Publication
(10) Pub. No.: US 2016/0339149 A1
(43) Pub. Date: Nov. 24, 2016

(54) METHOD OF PRODUCING CROSS-LINKED POLYVINYLALCOHOL-EXTRACELLUAR MATRIX COMPOSITE AND POLYVINYLALCOHOL-EXTRACELLUAR MATRIX COMPOSITE PRODUCED THEREBY

(71) Applicant: KOREA INSTITUTE OF SCIENCE AND TECHNOLOGY, Seoul (KR)

(72) Inventors: Kwi Deok PARK, Seoul (KR); In Gul KIM, Seoul (KR); Sang Heon KIM, Seoul (KR); Kang Won LEE, Seoul (KR); Min Tai Peter HWANG, Seoul (KR)

(21) Appl. No.: 15/156,068

(22) Filed: May 16, 2016

(30) Foreign Application Priority Data
May 18, 2015 (KR) 10-2015-0069126

(51) Int. Cl. A61L 27/40; A61L 27/16; A61L 27/36

(52) U.S. Cl. CPC A61L 27/40; A61L 27/3687; A61L 27/16; A61L 2430/20

(57) ABSTRACT

Provided are a method of producing a cross-linked PVA-ECM composite, and a PVA-ECM composite produced by using the method.

### CROSS-REFERENCE TO RELATED APPLICATION

This application claims the benefit of Korean Patent Application No. 10-2015-0069126, filed on May 18, 2015, in the Korean Intellectual Property Office, the disclosure of which is incorporated herein in its entirety by reference.

### BACKGROUND

1. Field

One or more embodiments relate to a method of producing a cross-linked polyvinylalcohol-extracellular matrix (PVA-ECM) composite and a PVA-ECM composite produced by using the method.

2. Description of the Related Art

A human biocompatible polymer material is widely applied to clinical trials as a means to diagnose, treat, and prevent diseases and is a basic material of artificial organs and artificial tissues that may be utilized to particularly alternate damaged or defunctionalized human tissues and organs. Examples of the artificial organs may include artificial hearts, artificial kidneys, cardiopulmonary machines, and artificial blood vessels; examples of the artificial tissues may include artificial joints, artificial bones, artificial skin, and artificial tendons; and examples of products for treatment may include dental materials, suture materials, and polymeric drugs. In addition, the polymer biomaterial is used in various fields, and a material with further improvement has been continuously required in the field that has not been resolved yet or has not been efficiently resolved until the present time.

All medical materials in addition to the polymer material necessarily need biocompatibility, and the biocompatibility may have two different meanings. Biocompatibility in a broad sense denotes having the desired function and safety with respect to a body at the same time, and biocompatibility in a narrow sense denotes biological safety with respect to a body, that is, having no toxicity and sterilizability. Thus, a biocompatibility polymer refers to a polymer that exhibits the desired function in the body and is not toxic as the material itself and sterilizable. However, if a cell surface receptor does not recognize a polymer surface during the cell attachment, a decrease in an effective value of the biocompatible polymer material is unavoidable. In recent years, studies for increasing cell affinity by treating the polymer surface with a natural polymer such as peptide, fibronectin, vitronectin, or laminin, which are related to cell attachment. These may be effective in attachment and amplification of cells to a certain degree but cannot be referred to as surface microenvironment close to biomimetic, which is a step for the actual cells to recognize as their environment. Therefore, developments of biocompatible structures and supports that are most close to the actual cell environment are needed. An extracellular matrix (ECM) obtained by culturing body tissues or cells is one of biomaterials that best realize the cellular microenvironment. In a conventional technology, a tissue ECM obtained by acellularizing cells from live allogenic or xenogenic tissues has small intestinal submucosa (SIS), urinary bladders (UBs), human amniotic membrane (HAM), or Achilles tendon as its main source; and may be used as a 3-dimensional support of various types based on its excellent physical properties (Korean Patent Registration No. 10-0715505). However, possibility of immune reaction of the material still exists and thus is limited to be applied to human bodies.

An ECM structure derived from a cell undergoes autologous cell culture and thus is free from the immune reaction, and a protein structure synthesized by amplification of the cell itself provides a physical topographical cue related to cell attachment, which is effective in migration and amplification of cells. Also, biogenic components such as collagen, fibronectin, or laminin in a matrix provide chemical microenvironment and thus may act preferably to be differentiated into particular cells. However, due to weak physical properties of being easily torn and broken despite the biological effects, the material may be difficult to be applied to the body and used as a 3-dimensional structure. Also, in terms of a conventional technology, attaching/detaching an ECM from a culture plate through an acellularizing process after the cell culture within the scope of not collapsing a shape of cells is not possible for now. The attaching/detaching of the ECM has only reached a level of physically scraping out cells by using a cell scraper. This ultimately destroys a cell's original structure and thus has a fatal weakness that a morphological advantage of the ECM may not be completely realized. If the ECM may be attached/detached in the same shape of a cell sheet (Japanese Patent No. 3043727), the ECM may be utilized in various forms and applied to various biological tissues.

In this regard, the present inventors have tried to develop a structure having an appropriate status to be applied to a body by attaching/detaching the matrix to/from a culture plate, wherein the matrix has a natural structure of the ECM maintained therein. As a result, the inventors have completed a PVA-ECM composite including a PVA that is detachable while an ECM is included therein by using physical cross-linking of a biocompatible PVA polymer.

### SUMMARY

One or more embodiments include a method of producing a cross-linked polyvinylalcohol-extracellular matrix (PVA-ECM).

One or more embodiments include a cross-linked PVA-ECM composite produced by using the method.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a view that schematically illustrating a process of coating a polyvinylalcohol (PVA) solution on an extracellular matrix (ECM) and preparing PVA-ECM gel that is physically cross-linked by using polyethylene glycol (PEG);

FIG. 2 shows a process of detaching the PVA-ECM gel, which is physically cross-linked by PEG, by holding one end with a forceps from a bottom of a 6-well plate (left) and a composite membrane thus obtained (right);

FIG. 3 shows the results of a surface of the PVA-ECM composite that is physically cross-linked by PEG analyzed with an optical microscope: an ECM-rich surface (left) and a PVA-rich surface (right);

FIG. 4 shows the results of observing the cross-linked PVA-ECM composite, which is immunofluorescent stained with respect to a fibronectin marker, with a fluorescent microscope: an ECM-rich surface (left) and a PVA-rich surface (right);

FIG. 5 is an fluorescent image of a fibroblast cell (NIH3T3) grown while being attached on the cross-linked PVA-ECM composite, Live & Dead stained, and observed with a fluorescent microscope;

FIG. 6 shows a patch of each group that has been implanted;

FIG. 7A shows the results of Masson's trichrome staining a mouse myocardial infarction site that includes a mesenchymal stem cell and to which a PVA-ECM composite is implanted;

FIG. 7B shows a percent of a fibrosis area in a left ventricle (LV) measured with respect to the image of FIG. 7A;

FIG. 8A shows the results of TTC staining with respect to each of myocardial segments;

FIG. 8B is a schematic view of a site at which cardinal segment has been performed;

FIG. 8C is a view showing an infarct size measured with respect to FIG. 8A;

FIG. 9A shows the results of immunofluorescent staining using an anti-SMA antibody and an anti-CD31 antibody with respect to arterioles; and

FIG. 9B is a view showing a density of the arterioles measured with respect to FIG. 9A.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to one aspect of the inventive concept, provided is a method of preparing a cross-linked PVA-ECM composite, wherein the method includes contacting a polyvinylalcohol (PVA) solution and an extracellular matrix (ECM) to obtain a mixture of the PVA and ECM; freezing and thawing the mixture to obtain a gelled PVA-ECM composite; and contacting the gelled PVA-ECM composite with a polyethylene glycol (PEG) solution to obtain a cross-linked PVA-ECM composite.

The method includes contacting of a PVA solution with an ECM to obtain a mixture of the PVA and ECM. The PVA is a water-soluble polymer that has a structural formula of [CH₂CH(OH)]n (where, n is an integer). A solvent of the PVA solution may be a solvent that can dissolve PVA. The solvent may have no toxicity or low toxicity to the human body. Examples of the solvent may include water, an aqueous buffer solution such as a phosphate buffered solution (PBS) or saline, and an organic solvent such as dimethylsulfoxide (DMSO) or dimethylformamide (DMF). A concentration of PVA in the PVA solution may be in a range of about 1 wt % to about 30 wt %, or, for example, about 1 wt % to about 20 wt %, about 1 wt % to about 10 wt %, about 5 wt % to about 20 wt %, about 10 wt % to about 30 wt %, about 5 wt % to about 30 wt %, about 10 wt % to about 20 wt %, or about 5 wt % to about 10 wt %. A weight average molecular weight of the PVA may be in a range of about 40,000 g/mol to about 500,000 g/mol, about 85,000 g/mol to about 200,000 g/mol, about 125,000 g/mol to about 190,000 g/mol, or about 40,000 to about 140,000 g/mol. An average degree of polymerization of the PVA may be in a range of about 1,150 to about 3,500, about 2,000 to about 3,500, or about 2,700 to about 3,500.

The ECM may be obtained by decellularizing biological tissues or a cultured cell layer. The ECM may be obtained by decellularizing a cultured fibroblast cell layer. The term “decellularization” refers to a process of removing cellular components from a tissue or a cell layer, which remains an ECM. The decellularization process may be performed by contacting an alkali, a DNase, or an RNase with the tissue or the cell layer. Thus, the ECM may have a structure of the cell layer or a cell group, for example, a 3-dimensional structure or a shape, maintained as it is. That is, the ECM may be a biomimetic that has a shape of the natural tissue or organ as it exists in an organism.

The contacting of the PVA and the ECM may be mixing the PVA and the ECM. The mixing may be performed while stirring the mixture. The mixture may be coating or adding the PVA on the ECM attached on a substrate. The PVA may percolate in to the ECM by the mixing. An amount of the PVA or ECM of the produced PVA-ECM composite may vary in a thickness direction depending on a concentration of the PVA and a time of the contacting. For example, an ECM amount at one surface of the produced PVA-ECM composite in a thickness direction may be higher than the amount at a different surface. Therefore, the produced PVA-ECM composite may have a surface having a high ECM amount and a surface having a high PVA amount. In this regard, the contacting includes controlling a PVA concentration and a contact time so that a PVA-ECM composite with a surface having a high ECM amount and a surface having a high PVA amount may be formed.

The method includes freezing and thawing the mixture to obtain a gelled PVA-ECM composite. The freezing and thawing may be performed at least once, for example, twice or more. The freezing and thawing may be performed twice or more, 4 times or more, 6 times or more, 10 times or more, 15 times or more, 20 times or more, or 40 times or more. The freezing and thawing may be performed in a range of once to 20 times, once to 15 times, once to 12 times, once to 5 times, twice to 20 times, 5 times to 15 times, once to 12 times, or once to 5 times. The freezing may be performed at a temperature in a range of about −60° C. to about −0° C., about −60° C. to about −1° C., about −60° C. to about −10° C., about −60° C. to about −20° C., about −50° C. to about −25° C., about −35° C. to about −30° C., about −50° C. to about −1° C., about −40° C. to about −1° C., about −30° C. to about −1° C., or about −30° C. to about −0° C. The freezing may be preformed for time that allows the mixture to be sufficiently frozen, which may be, for example, in a range of about 1 second to about 12 hours, about 1 hour to about 12 hours, about 3 hours to about 8 hours, or about 4 hours to about 5 hours. The thawing may be performed at a temperature and time appropriate for thawing the frozen mixture. The thawing may be performed at a temperature in a range of about 10° C. to about 60° C., about 25° C. to about 45° C., about 30° C. to about 35° C., or about 25° C. to about 60° C., or at room temperature. The thawing time may be in a range of about 1 hour to 6 hours, about 2 hours to about 5 hours, or about 3 hours to about 4 hours.

The method may include contacting the gelled PVA-ECM composite with a PEG solution to obtain a cross-linked PVA-ECM composite. The PEG may be a polyether compound that has an oxyethylene moiety. The PEG may be represented by —(O—CH₂—CH₂)n-, or, for example, H—(O—CH₂—CH₂)n-OH (where, n is an integer). In this formula, —OH at the end may be substituted with a C1-C6 alkyl group (e.g., a methyl group). The PEG may be a low molecular weight oligomer type having a weight average molecular weight of about 1,000 g/mol or lower, or, for example, in a range of about 100 g/mol to about 1,000 g/mol, about 300 g/mol to about 1,000 g/mol, about 400 g/mol to about 1,000 g/mol, about 300 g/mol to about 600 g/mol, or about 400 g/mol to about 600 g/mol. A solvent of the PEG solution may be a solvent that can dissolve PEG. The solvent may have no toxicity or low toxicity to the human body. Examples of the solvent may include water, an aqueous buffer solution such as a phosphate buffered solution (PBS) or saline, and an organic solvent such as dimethylsulfoxide (DMSO) or dimethylformamide (DMF).

The contacting of the gelled PVA-ECM composite and the PEG solution may be mixing the gelled PVA-ECM composite and the PEG solution. The mixing may be performed while stirring the mixture. The mixture may be coating PEG on the gelled PVA-ECM composite attached on a substrate. The PEG may percolate in to the gelled PVA-ECM composite by the mixing and physically cross-linking the PVA-ECM composite. Due to the cross-linking, a cross-linked PVA-ECM composite having an increased tensile strength than that of the PVA-ECM composite may be produced. A tensile strength of the cross-linked PVA-ECM composite may be in a range of about 0.1 kgf/mm² to about 0.8 kgf/mm², or, for example, about 0.3 kgf/mm² to about 0.8 kgf/mm², about 0.5 kgf/mm² to about 0.8 kgf/mm², about 0.7 kgf/mm² to about 0.8 kgf/mm², about 0.1 kgf/mm² to about 0.5 kgf/mm², or about 0.1 kgf/mm² to about 0.3 kgf/mm².

The cross-linked PVA-ECM composite may have a tensile strength to a degree that the composite is not torn when one end is forced in a gravity direction while holding the other end. The cross-linked PVA-ECM composite may be hydrogel. The cross-linked PVA-ECM composite may be in a wet form or a dry form. The cross-linked PVA-ECM composite in a wet form may be in a dry form by being dried or, for example, freeze-dried. The cross-linked PVA-ECM composite may have any shape. A shape of the cross-linked PVA-ECM composite may be determined by a shape of the substrate, on which the ECM is to be attached. The cross-linked PVA-ECM composite may have a sheet-like shape. The contact may be performed for a period of time and at a temperature sufficient for the PEG to induce the cross-linking of the PVA and ECM. The contact may be performed at a temperature in a range of about 5° C. to about 50° C., about 10° C. to about 40° C., about 20° C. to about 30° C., or about 3° C. to about 50° C., or at room temperature. The contact time may be in a range of about 1 minute to about 60 minutes, about 5 minutes to about 50 minutes, about 10 minutes to about 30 minutes, or about 30 minutes to about 60 minutes.

The method may include culturing cells on a surface of a culture dish and removing cellular components from the culture cell layer to prepare an ECM attached on the surface of the culture dish before the contacting of the PVA solution with the ECM. In the culturing of the cells on a surface of the culture dish, the cells may be cells of a mammal, for example, a human, a mouse, a pig, a cow, or a sheep. The cells may be fibroblast cells, chondrocytes, osteoblasts, endothelial cells, myoblasts, smooth muscle cells, hepatocytes, neural cells, cardiomyocytes, or interverteveral disc cells. The culture dish may be a culture dish having a surface that is generally used in cell culture. The culture cell may be in the form of a plate having at least one well, such as 6 wells or 96 wells. The culturing may be performed by selecting a medium appropriate for the selected cells among mediums generally used in culturing cells of a mammal. The surface of the culture dish where the cells grow may have any shape or may be, for example, a flat surface or 3-dimensionally shaped.

The method may further include washing the cross-linked PVA-ECM composite to remove the PEG after the contacting of the gelled PVA-ECM composite with the PEG solution. A liquid used in the washing may have no toxicity or low toxicity to the human body. The wash liquid may be water, an aqueous buffer solution such as a phosphate buffered solution (PBS) or saline, or an organic solvent such as dimethylsulfoxide (DMSO) or dimethylformamide (DMF).

In the method, the contacting of the PVA solution with the ECM to obtain a mixture of the PVA and ECM may be performed by contacting the PVA solution with the ECM attached on the culture dish surface to obtain a mixture of the PVA and ECM. The culture dish may be a flat-surfaced incubator that has a 3-dimensional shape.

The method may further include physically detaching the cross-linked PVA-ECM composite from the culture dish surface. The physically detaching may be performed by, for example, pulling the cross-linked PVA-ECM composite in a direction away from the culture dish surface while holding a part of a surface of the cross-linked PVA-ECM composite. The method may not include a chemical cross-linking process. Also, the cross-linked PVA-ECM composite may be used as it is without additional molding.

According to another aspect of the inventive concept, provided is a cross-linked PVA-ECM composite produced by using the method described above. The cross-linked PVA-ECM composite may not substantially include PEG. A tensile strength of the cross-linked PVA-ECM composite may be in a range of about 0.1 kgf/mm² to about 0.8 kgf/mm², or, for example, about 0.3 kgf/mm² to about 0.8 kgf/mm², about 0.5 kgf/mm² to about 0.8 kgf/mm², about 0.7 kgf/mm² to about 0.8 kgf/mm², about 0.1 kgf/mm² to about 0.5 kgf/mm², or about 0.1 kgf/mm² to about 0.3 kgf/mm². The cross-linked PVA-ECM composite may have a shape of a sheet or a layer or may have a 3-dimensional composite structure. The cross-linked PVA-ECM composite may have a PVA-rich surface and an ECM-rich surface. The PVA-rich surface and the ECM-rich surface may be on different surfaces, i.e., opposite sides. That is, the PVA-ECM composite may have different amounts of PVA or ECM in the thickness direction. The cross-linked PVA-ECM composite may have cells grown on the ECM-rich surface and thus may be used as an implant for biological tissue regeneration. Examples of the biological tissue may include bones, tendons, ligaments, vessels, urinary organs, and skin. Thus, the PVA-ECM composite may have a shape of a tissue or an organ of bones, tendons, ligaments, vessels, urinary organs, hearts, or skin. The implant may be a patch that is used to apply medication on skin. For example, the implant may be a wound healing patch, a cardiac patch, or a patch for treating diabetic foot ulcers, or a membrane for preventing adhesion.

As used herein, the PEG has been described as an example of a polyalkylene ether compound having an oxyalkylene moiety. Thus, as used herein, the PEG may be substituted with a polyalkylene ether compound having an oxyalkylene moiety. The polyalkylene ether compound may be represented by —(O—R₁)ₙ—, or, for example, H—(O—R₁)ₙ—OH. In these formulae, n is an integer, Ri is a C1-C6 alkyl group, or, for example, a methyl group, an ethyl group, a propyl group, a butyl group, a pentyl group, or a hexyl group.

Hereinafter, one or more embodiments will be described in detail with reference to the following examples. However, these examples are not intended to limit the scope of the one or more embodiments.

### EXAMPLE 1

1. Preparation of ECM

Cells were inoculated at a density of 5×10⁴ cells/cm² in each well of a 6-well plate (CorningTM) for culturing cells, and the cells were cultured in an incubator under conditions of 37° C. and 5% CO₂ for 5 days to 7 days. The cultured cells were human lung fibroblast (WI38) which were cultured in a DMEM (Gibco: Cat.no. 11995-073)/10% FBS medium. After the culturing was completed, in a 100% confluent state, the medium was removed, and the cells were washed with 3 ml of PBS once, followed by adding 1 ml of a decellularization solution (including 0.25% of Triton X-100 and 10 mM of NH₄OH in PBS). The cells were immersed in the decellularization solution at room temperature for 2 minutes, and then the decellularization solution was removed. Next, the cells were immersed in a 50 unit/ml DNase and 50 ug/ml RNase solution (PureLinkTM RNase A: Invitrogen: Cat. no. 12091-039) and incubated in a 37° C. incubator for 2 hours. As a result, an ECM without cellular components was obtained while being attached on a substrate in the form of a membrane. 3 ml of PBS was added to the wells to wash the ECM 3 times, and a PBS solution containing 0.1 M of glycine was added thereto. Then, the cells were stored at 4° C. until use.

2. Preparation of Cross-Linked PVA-ECM Composite

PVA (Sigma Aldrich) having a weight average molecular weight of 140,000 g/mol in the form of a powder was added to deionized water in a glass container to prepare a 8 wt % PVA aqueous solution. The aqueous solution was placed in an autoclave and heat-treated. The heat treatment was performed at 120° C. and 30 Ib/int for 1 hour. A PVA aqueous solution obtained as a result was transparent, phlegmatic, and homogenous.

Next, PBS was added to the 6-well plate to wash the ECM once, where the ECM prepared in Clause 1 was attached in the form of a membrane in each of the wells, and then water was completely removed therefrom. 1 ml of the PVA aqueous solution prepared as described above was evenly added on to the ECM in each of the wells. Since a thickness of the cross-linked PVA-ECM composite differs depending on an amount of the added PVA aqueous solution, and thus the amount of the PVA aqueous solution may be controlled. The 6-well plate was placed on a stirrer and remained thereon at 50 rpm for 5 minutes so that the PVA aqueous solution was well percolated in the ECM. Then, the resultant was frozen at -20° C. for 12 hours and thawed at room temperature for 30 minutes to primarily prepare a PVA-ECM composite that was formed by weak physical cross-linking between the PVA and ECM. The PVA-ECM composite was in the form of a soft membrane and is also referred to as ‘cyrogel’. The cyrogel did not detach from the bottom when it was pulled from the bottom while holding one side and was easily torn.

Then, 600 uL of PEG (having a molecular weight of 400 g/mol) was added on to the cyrogel to sufficiently wet the whole cyrogel. The adding of the PEG was performed at room temperature, and the resultant was remained at room temperature for about 5 minutes thereafter. The prepared cross-linked PVA-ECM gel was detached from the bottom while holding one side by the PEG thus prepared, and the gel was not torn during the detachment. It is deemed that this is because the PEG was percolated in to the whole gel by self diffusion between PVA molecules, which rapidly increased crystallinity of the PVA chains, and thus the chains of the PVA were fixed to each other. Therefore, the term “cross-linked” in the expression “cross-linked by PEG” used herein may denote an increase in a crystalline region induced by PEG, changes in physical properties according to the increase, wherein the changes may include compacting PVA or ECM chains, physical secondary cross-linking, and increasing opacity of the composite. However, the scope of the inventive concept is not limited to a particular mechanism. As a result, the cross-linked PVA-ECM physically obtained by PEG may not be easily torn and has good elasticity.

Subsequently, the prepared PVA-ECM gel was washed with 50 ml PBS several times for about 5 hours to about 8 hours. Here, the PEG, as an aqueous polymer that served as a secondary cross-linking agent, was dissolved in PBS and thus was removed. Then, the PVA-ECM gel was freeze-dried for 2 days at -50° C. and 5 torr to prepare the gel in a dry form. The gel in the dry form may be preserved for a long time via vacuum packing and may be easily migrated and applied to a body. Alternatively, the washing may be performed while the PVA-ECM gel is attached on a surface of the container. After the washing, the PVA-ECM gel may be detached from the surface.

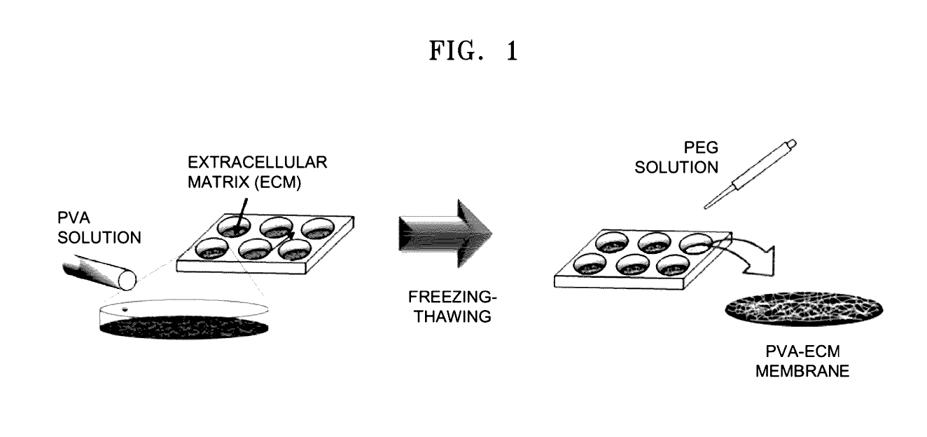

FIG. 1 is a view that schematically illustrating a process of coating a PVA solution on an ECM and preparing PVA-ECM gel that is physically cross-linked by PEG.

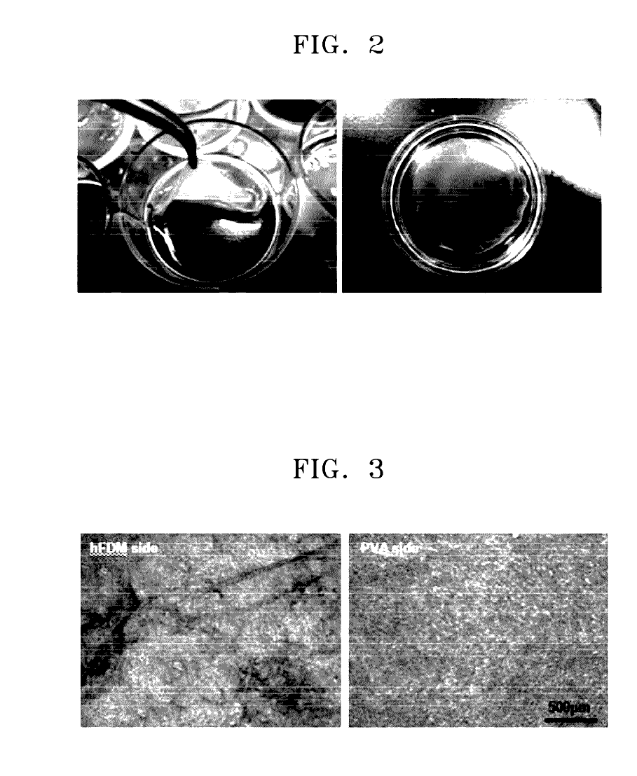

FIG. 2 shows a process of detaching the PVA-ECM gel, which is physically cross-linked by PEG, by holding one end with a forceps from a bottom of a 6-well plate (left) and a composite membrane thus obtained (right).

FIG. 3 shows the results of a surface of the PVA-ECM composite that is physically cross-linked by PEG analyzed with an optical microscope: an ECM-rich surface (left) and a PVA-rich surface (right). In FIG. 3, hFDM denotes a human fibroblast-derived matrix.

3. Fibronectin-Coated PVA-PEG Gel

A PVA-PEG gel coated with fibronectin was prepared as a control group for an animal experiment of the PVA-ECM composite. In particular, PVA having a weight average molecular weight of 140,000 g/mol in the form of a powder was added to deionized water to prepare a 8 wt % PVA aqueous solution. Then, the resultant was frozen at -20° C. for 12 hours and thawed at room temperature for 30 minutes to primarily prepare a physically cross-linked PVA cyrogel. Here, the PVA cyrogel did not have an ECM. Next, the PVA cyrogel underwent freezing-thawing, 600 uL of PEG (having a molecular weight of 400 g/mol) was added on to the PVA cyrogel to sufficiently wet the whole cyrogel. Thereafter, the resultant was remained at room temperature for about 5 minutes. Subsequently, the PVA-PEG gel after secondary cross-linking was washed with 50 ml of PBS several times for about 5 hours to about 8 hours through deionized water. A fibronectin coating on the prepared PVA-PEG gel was performed by immersing the PVA-PEG gel in a fibronectin aqueous solution having a concentration of 50 μg/ml and incubating the gel for 1 day at 37° C. This is generally marked as “PVA-FN”.

4. Surface Characteristic Analysis by Fibronectin Immunofluorescence Staining

In order to confirm whether a component of the ECM is well bound to the PVA-ECM composite physically cross-linked by PEG prepared in Clause 2, presence of fibronectin, which is an ECM component, on the cross-linked PVA-ECM composite was confirmed by immunofluorescence staining with respect to a fibronectin marker.

In particular, the ECM prepared in Clause 1 after removing the cellular components therefrom was washed with PBS, fixed on a slide at room temperature by using 4% paraformaldehyde for 15 minutes, additionally fixed with cold acetone at -20° C. for 10 minutes, and completely dried at room temperature. The dried sample was washed with PBS, and non-specific protein binding was suppressed by a 3% bovine serum albumin (BSA) solution in the PBS. Next, as a primary antibody, a mouse monoclonal anti-fibronectin IgG antibody was diluted in a 1% BSA solution at a ratio of 1:50, and the sample was added thereto and preserved at 4° C. for 12 hours. Then, an Alexa Flour 488-conjugated goat anti-mouse IgG (Invitrogen), as a secondary antibody, was diluted and added at a concentration ratio of 1:200, and the mixture was allowed to react at room temperature for about 1 hour. The resultant was washed with PBS.

Thereafter, PVA was added thereto in the same manner described in Clause 2 to form cyrogel, and PEG was added thereto and allowed to react to finally obtain fibronectin-containing PVA-ECM gel. Then, the cross-linked PVA-ECM gel was separated by using a forceps and attached on a cover glass to observe fibronectin by using a confocal fluorescence microscope (Olympus BX41).

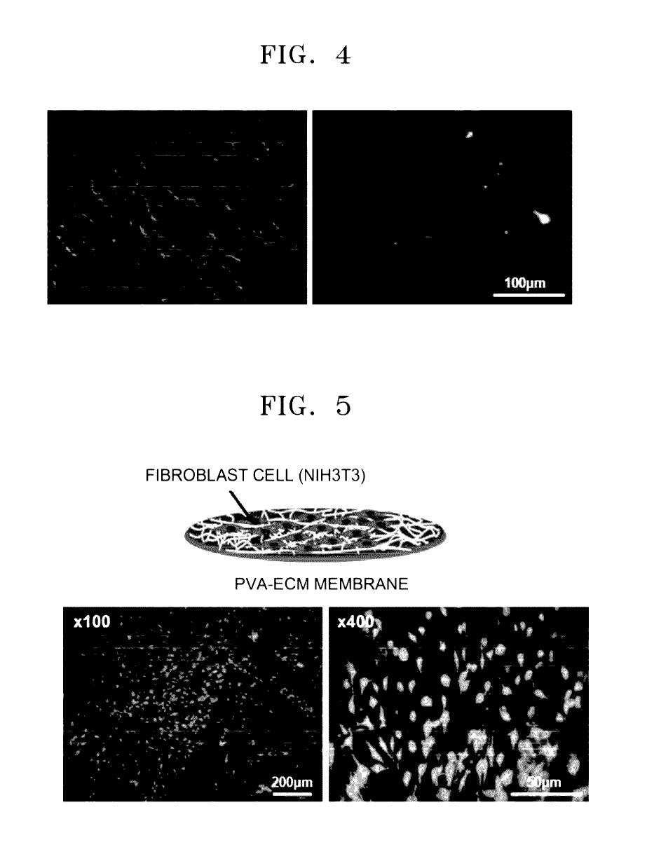

FIG. 4 shows the results of observing the cross-linked PVA-ECM composite, which is immunofluorescent stained with respect to a fibronectin marker, with a fluorescent microscope: an ECM-rich surface (left) and a PVA-rich surface (right). As shown in FIG. 4, fibronectin stained in green fluorescence was confirmed only on the ECM-rich surface (left), and no fluorescence staining was observed on the PVA-rich surface (right).

5. Cell Aattachment and Amplification on Cross-Linked PVA-ECM Xomposite

In each well of the 6-well plate, the cross-linked PVA-ECM composite prepared in Clause 2 was placed in a manner that exposes the ECM-rich surface and allows the PVA-rich surface to contact the bottom, cells were inoculated thereto at a concentration of 5×10⁴ cells/cm² and cultured to evaluate attachment and amplification of the cells. The ECM used herein was a human lung fibroblast-derived matrix (hFDM). Also, the inoculated cells were fibroblast cells (NIH3T3). The medium was DMEM/10% FBS.

The cells were inoculated on the cross-linked PVA-ECM composite as described in Clause 2, and a fluorescent stained image for cell morphology was observed by using Live & Dead staining within 72 hours after the inoculation. The cell growth was measured by evaluating the cell number change in 4 days by using the CCK-8 assay (Cell Counting Kit-8, Dojindo, Japan).

FIG. 5 is an image of a fibroblast cell (NIH3T3) grown while being attached on the cross-linked PVA-ECM composite, Live & Dead stained, and observed with a fluorescent microscope. In FIG. 5, an upper part schematically illustrates implantation of cells to the cross-linked PVA-ECM composite, and a lower part shows fluorescent microscope images of the attached cells (left ×100, right ×400). As shown in FIG. 5, the hFDM cells efficiently grew on the PVA-ECM composite.

6. Measurement of Tensile Strength of Cross-Linked PVA-ECM Composite

4 groups of samples of the PVA-ECM composites prepared in Clauses 1 and 2 having different molecular weights and concentrations and one sample (group 5) of PVA gel free of PEG (which underwent 3 times of freezing and thawing) were prepared in a shape of a dumbbell based on ASTM D412. Tensile strengths of the samples were measured at a rate of cross-head 20 mm/min, and the results are shown in Table 1.

TABLE 1

| | | Tensile | Tensile | Elongation |
|---|---|---|---|---|
| | | modulus | strength | percentage |
| No. | Test group | (Mpa) | (kgf/mm²) | (%) |
| 1 | PVA(MW140000/ | 0.37 ± 0.05 | 0.49 ± 0.07 | 486.4 |
| | 8 wt %)/PEG | | | |
| 2 | PVA(MW140000/ | 0.43 ± 0.09 | 0.26 ± 0.14 | 533.6 |
| | 5 wt %)/PEG | | | |
| 3 | PVA(MW80000/ | 0.31 ± 0.12 | 0.32 ± 0.03 | 367.8 |
| | 8 wt %)/PEG | | | |
| 4 | PVA(MW80000/ | 0.23 ± 0.15 | 0.23 ± 0.09 | 412.2 |
| | 5 wt %)/PEG | | | |
| 5 | Pure PVA gel | 0.37 ± 0.08 | 0.15 ± 0.07 | 180.5 |

In Table 1, the ‘pure PVA gel’ was prepared as follows. First, the PVA (Sigma Aldrich) having a weight average molecular weight of 140,000 g/mol in the form of a powder was added to deionized water in a glass container to prepare a 8 wt % PVA aqueous solution. The aqueous solution was placed in an autoclave and heat-treated. Autoclaving is used to melt as well as to sterilize PVA. The heat treatment was performed at 120° C. and 30 Ib/int for 1 hour. A PVA aqueous solution obtained as a result was transparent, phlegmatic, and homogenous. Next, 1 ml of the PVA aqueous solution thus obtained was evenly added to empty wells of a 6-well plate, to which the ECM prepared in Clause 1 was not attached. Then, the plate was frozen at -20° C. for 12 hours and thawed at room temperature for 30 minutes to primarily prepare PVA gel, and the PVA gel was further frozen and thawed twice to prepare pure PVA hydrogel in the form of a soft membrane that is not easily torn.

As shown in Table 1, it was confirmed that the PVA-ECM gel was significant improved in terms of tensile strength and elongation percentage according to a molecular weight of the PVA compared to those of pure PVA gel, but there was not much difference in terms of the elastic coefficients.

7. Evaluation on effect as cardiac patch by using animal model with myocardial infarction (in vivo)

In order to evaluate treatment effects of the PVA-ECM composites prepared in Clauses 1 and 2 as a cardiac patch, white mice having acute myocardial infarction were used as an animal model. Here, the term “cardiac patch” refers to a PVA-ECM composite in the form of a patch prepared for regeneration of cardiac muscles that are damaged. The patch may be inoculated with a human mesenchymal stem cell (hMSC).

The white mice (Sprague-Dawley; Samtako Bio, Osan, Korea) used in the experiment were male of about 10-week old having a body weight in a range of about 250 g to about 300 g. The white mice animal model having acute myocardial infarction was prepared as follows. First, 10 mg/kg of tiletamine/zolazepam (Zoletil 50) and 2 mg/kg of 2% xylazine hydrochloride (Rumpun) were mixed, and the mixture was injected into a lower abdomen of the white mouse to perform anesthesia. The anesthetized white mouse was placed immovable on an operating table and provided with artificial respiration by connecting to a ventilator through a respiratory tract of the mouse. An under-skin layer was delaminated, a region between the No. 5 costal bone and the No. 6 costal bone was punctured to open a thoracic cavity. The region between the costal bones was opened by using a fixing device to secure a vision so that a heart and a lung may be well seen. A pericardinum was removed, and a left anterior descending artery of about 0.3 mm distance at a junction was ligated. Whether the mouse had acute myocardial infarction was confirmed by observing pallidness in the left ventricle of the hear with the naked eye after the ligation.

The experimental groups included a group of PVA-FN (prepared in Clause 3 of Example) patch coated with fibronectin (hereinafter, also referred to as “FN+hMSC”) and a PVA-ECM patch group to which a human fibroblast-derived ECM (hFDM) is attached (hFDM+hMSC) (which is prepared according to Clauses 1 and 2 of Example). Here, each of the patches was prepared into a circular membrane having a diameter of about 8 mm and a thickness of about 100 ₛum, more or less. Also, hMSC was inoculated on each of the experimental patches at a cell concentration of 2×10⁵/200 μL (PBS) one day before implantation and cultured for one day. The implantation of the patch to a myocardial infarction site was performed by implanting each of the patches onto the myocardial infarction site in the opened heart and fixing the patch by using fibrin glue (Tissel™).

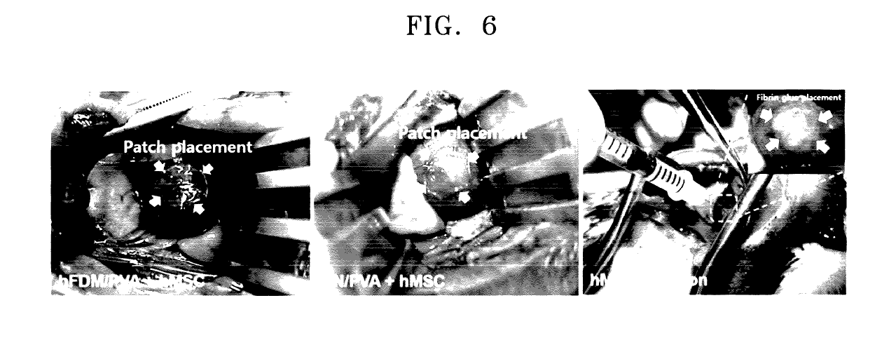

Also, as another comparison group representing a conventional method of using a cell treating agent, 200 μl of hMSC at a concentration of 1×10⁶/ml was simply injected to a boundary of the myocardial infarction site (hMSC injection), and white mice having myocardial infarction not treated with anything were used as a control group. Also, fibrin glue (TisselTM) was applied on the site to prevent detachment of the implanted patch. FIG. 6 shows the state of the implanted patch of each of the groups.

8. Histological Analysis (Masson's Trichrome Staining)

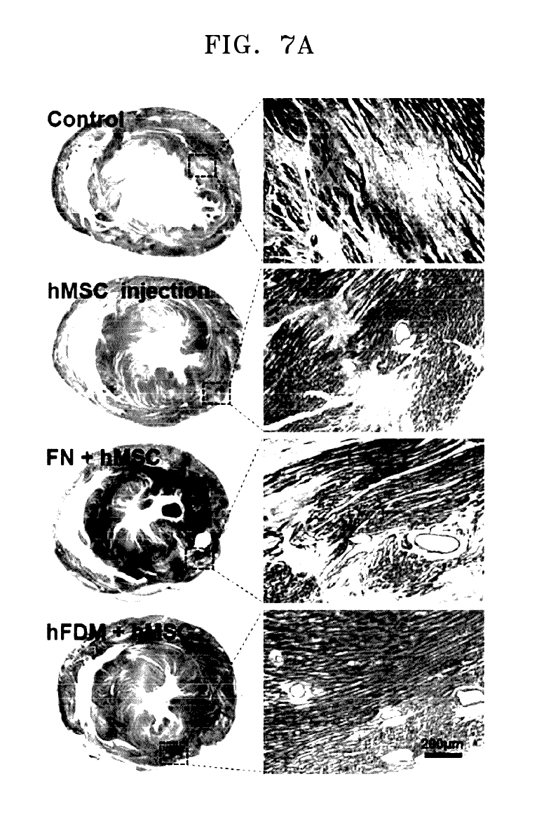

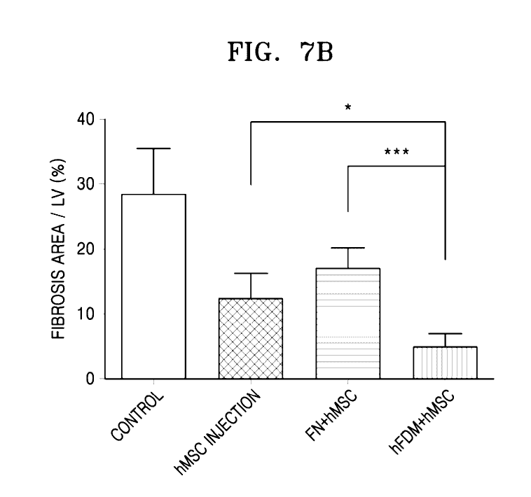

The cardiac patch inoculated with MSC as in Clause 6 was implanted to a myocardial infarction site, some mice were sacrificed after 3 weeks, heart tissues were obtained by perfusion, and the tissues were fixed in a 10% neutral formalin solution for 1 day. The fixed heart tissues were sectioned at a thickness of 5 pm in a vertical direction, Masson's trichrome stained, and observed by using an optical microscope to confirm a degree of fiberization of the myocardial tissues. The results are shown in FIGS. 7A and 7B.

FIG. 7A shows the results of Masson's trichrome staining a mouse myocardial infarction site that includes a mesenchymal stem cell and to which a PVA-ECM composite is implanted. FIG. 7B shows a percent of a fibrosis area in a left ventricle (LV) measured with respect to the image of FIG. 7A. In FIG. 7A, blue represents a fibrosis site, and red represents myocardial cells. As shown in FIG. 7A, a low degree of fibrosis was observed from the patch group bound with fibroblast cell-derived ECM (hFDM), but a relatively large area of myocardial tissues were found. This is because paracrine effects of the MSC were maximized from the ECM which suppressed occurrence of apoptosis in the left ventricle, and thus ultimately a thickness of the left ventricle could have been maintained. Also, it may be known that muscles were organized at a high level in the patch group bound with hFDM compared to those of other experimental groups. As shown in FIG. 7B, the fibrosis area in the hFDM+hMSC group was the smallest.

9. Tetrazolium Chloride (TTC) Staining

The cardiac patch inoculated with MSC as in Clause 6 was tetrazolium chloride (TTC) stained to measure and analyze a size of an acute myocardial infarction site 3 weeks after implanting the patch on to the myocardial infarction site. The myocardial segment was immersed in 2,3,5 triphenyltetrazolium chloride (TTC; Muto, Japan) at a concentration of 2% and incubated in a water tub at 37° C. for 30 minutes and fixed in 10% formalin. A relative myocardial infarction site of the TTC stained segment was measured and analyzed by using an automatic image analyzer and shown in a percent (%).

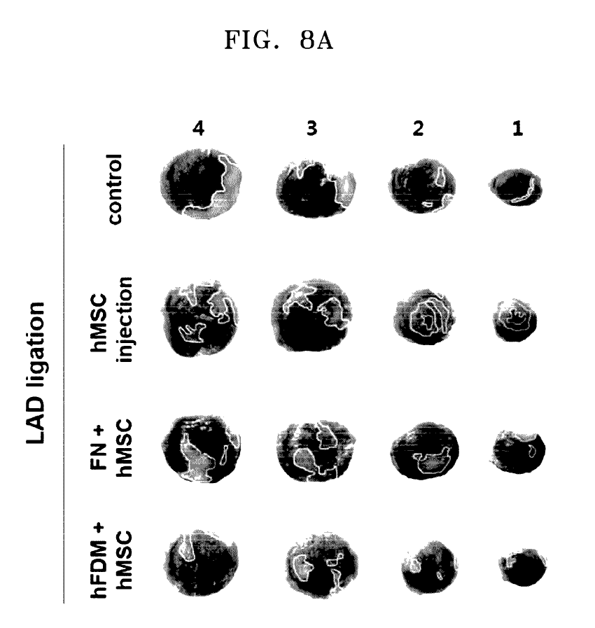

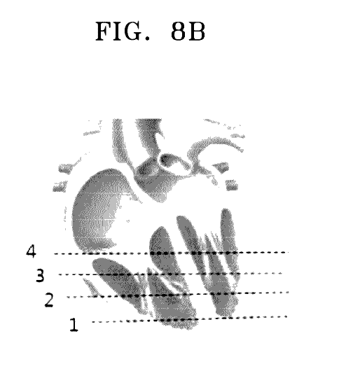

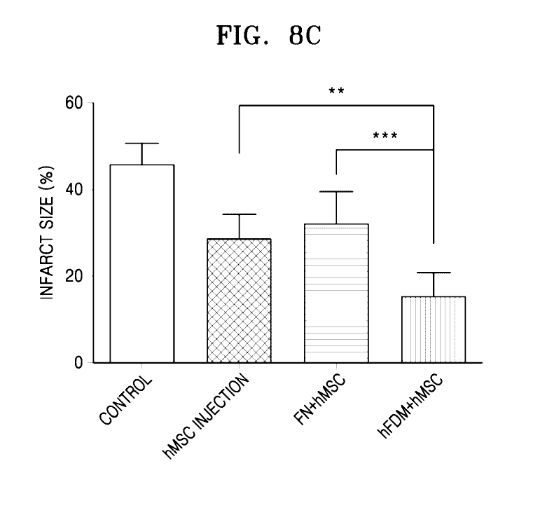

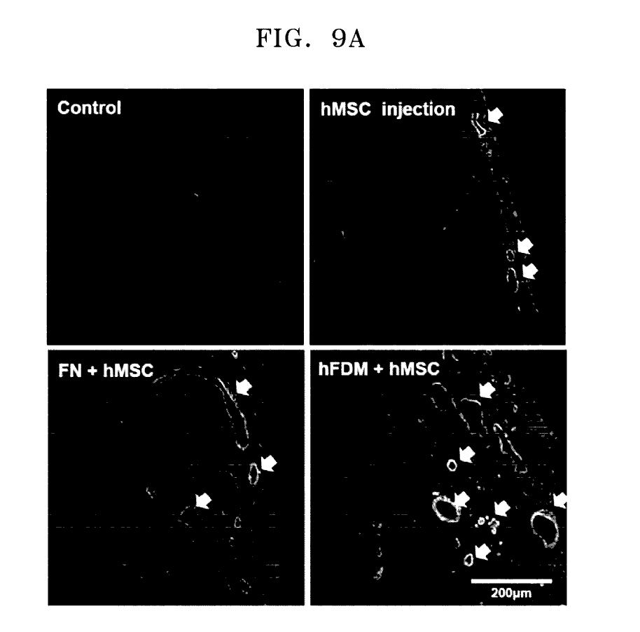

The results are shown in FIGS. 8A, 8B, and 8C. FIG. 8A shows the results of TTC stain with respect to the each of the myocardial segments. In FIG. 8A, 1, 2, 3, and 4 show cross-sections of the segments that were cut along dotted lines 1, 2, 3, and 4 as shown in FIG. 8B, and LAD ligation represents left anterior descending artery ligation. As shown in FIG. 8A, a white part that is not stained red represents a fibrosis area, and it may be confirmed that infarction of myocardial tissues in the myocardial infarction model group, which is a control group, had occurred to a measurable degree. On the other hand, infarction sites reduced in the experimental groups in general, and, particularly, it may be confirmed that the fibrosis area significantly decreased in the patch group bound with hFDM compared to those of the other two experimental groups. FIG. 8B a schematic view of a site at which cardinal segment has been performed. FIG. 8C is a view showing an infarct size measured with respect to FIG. 8A. The infarct size represents an area of the white part in FIG. 8A. As shown in FIG. 8C, the infarct size was the smallest in the hFDM+hMSC group.

10. Analysis on Vessel (Arteriole) Formation

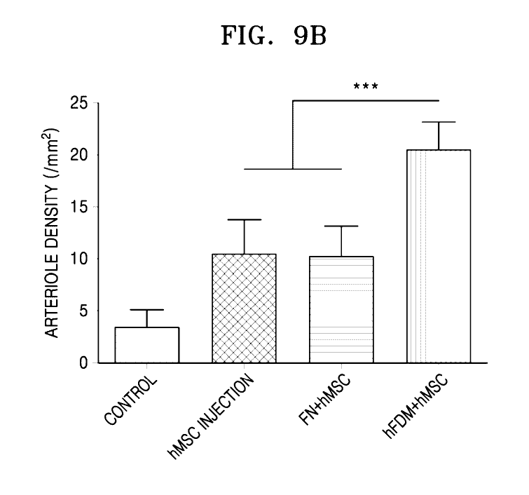

Arteriole regeneration was observe at an infraction site to which the patch was implanted 3 weeks after implanting the cardiac patch inoculated with MSC as in Clause 6 on to the myocardial infarction site. In this regard, immunofluorescent staining was performed by using an anti-SMA antibody that specifically binds to a-smooth muscle actin (SMA) in vascular endothelial smooth muscle and an anti-CD31 antibody that binds to CD31, which is specific to blood vessels. The results are shown in FIGS. 9A and 9C. FIG. 9A shows the results of immonofluorescent staining using an anti-SMA antibody and an anti-CD31 antibody with respect to arterioles. In FIG. 9A, arrows indicate arterioles, and blue represents nuclei of tissue cells. FIG. 9B is a view showing a density of the arterioles measured with respect to FIG. 9A. As shown in FIG. 9A, the control group had almost no blood vessels induced around the infarction site, but the experimental groups all had significant numbers of arterioles. In particular, a large number of arterioles were observed in the hFDM+hMSC group. As shown in FIG. 9B, the hFDM+hMSC group had the highest arteriole density.

As confirmed in the examples described above, the PVA-ECM composite prepared according to one or more embodiment of the present disclosure produces significant effects in terms of improving tissue regeneration.

As described above, according to one embodiment, a method of producing a cross-linked PVA-ECM composite may effectively provide a cross-linked PVA-ECM composite and a 2-dimensional matrix structure that is in the state capable of handling an ECM derived from cells as it is detached from a bottom surface.

According to another embodiment, the cross-linked PVA-ECM composite has excellent elongation percentage and restoring ability and thus may be used in the mechanobiology field.

According to another embodiment, the cross-linked PVA-ECM composite may be used in tissue regeneration for basic regenerative medicine and may be utilized as a treatment material of in various patch types.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the inventive concept as defined by the following claims.

What is claimed is:

1. A method of producing a cross-linked PVA-ECM composite, the method comprising:
contacting a polyvinylalcohol (PVA) solution and an extracellular matrix (ECM) to obtain a mixture of PVA and ECM;
freezing and thawing the mixture to obtain a gelled PVA-ECM composite; and
contacting the gelled PVA-ECM composite with a polyethylene glycol (PEG) solution to obtain a cross-linked PVA-ECM composite.

2. The method of claim 1, wherein the PVA solution is a PVA in water, dimethylsulfoxide (DMSO), phosphate buffered saline (PBS), or saline.

3. The method of claim 1, wherein a concentration of PVA in the PVA solution is in a range of about 1 wt % to about 30 wt %.

4. The method of claim 1, wherein a weight average molecular weight of the PVA is in a range of about 40,000 g/mol to about 500,000 g/mol.

5. The method of claim 1, wherein the ECM is obtained by decellularizing a biological tissue or a cultured cell layer.

6. The method of claim 1, wherein the ECM is obtained by decellularizing a cultured fibroblast cell layer.

7. The method of claim 1, wherein the freezing and thawing is performed at least once.

8. The method of claim 1, wherein the freezing is performed at a temperature in a range of about −20° C. to about −60° C.

9. The method of claim 1, wherein the freezing is performed for about 1 second to about 12 hours.

10. The method of claim 1, wherein a weight average molecular weight of the PEG is in a range of about 100 g/mol to about 1,000 g/mol.

11. The method of claim 1, wherein the PEG is a low molecular weight PEG of an oligomer type.

12. The method of claim 1, comprising culturing a cell on a surface of a culture dish and removing a cell component from the cultured cell layer to prepare an ECM attached on the surface of the culture dish before the contacting the PVA solution with the ECM.

13. The method of claim 12, wherein the surface of the culture dish where the cell grows is a flat surface.

14. The method of claim 1, wherein the contacting of the gelled PVA-ECM composite with the PEG solution is performed at a temperature in a range of about 5° C. to about 50° C.

15. The method of claim 1, wherein the contacting of the gelled PVA-ECM composite with the PEG solution is performed for about 1 minute to about 60 minutes.

16. The method of claim 1 further comprising washing the cross-linked PVA-ECM composite to remove the PEG after the contacting of the gelled PVA-ECM composite with the PEG solution.

17. The method of claim 12, wherein the contacting of the PVA solution with the ECM to obtain a mixture of the PVA and the ECM comprises contacting the PVA solution with the ECM attached on the surface of the culture dish to obtain a mixture of PVA and ECM.

18. The method of claim 17 further comprising physically separating the cross-linked PVA-ECM composite from the surface of the culture dish.

19. The method of claim 1, wherein the cross-linked PVA-ECM composite is hydrogel.

20. The method of claim 1, wherein the cross-linked PVA-ECM composite has a sheet shape.

21. The method of claim 1, wherein a tensile strength of the cross-linked PVA-ECM composite is in a range of about 0.1 kgf/mm² to about 0.8 kgf/mm².

22. A cross-linked PVA-ECM composite produced by using the method of claim
